(19) United States
(12) Patent Application Publication
(10) Pub. No.: US 2025/0003907 A1
(43) Pub. Date: Jan. 2, 2025

(54) DEVICE AND METHOD FOR ELECTROMAGNETICALLY CHARACTERIZING A MEDIUM USING A CONTACTLESS RESONATOR, AND ASSOCIATED OBJECT AND RESONATOR

(71) Applicants: UNIVERSITE PARIS-SACLAY, GIF-SUR-YVETTE (FR); CENTRE NATIONAL DE LA RECHERCHE SCIENTIFIQUE, PARIS (FR); CY CERGY PARIS UNIVERSITÉ, Cergy-Pontoise Cedex (FR)

(72) Inventors: Alexiane PASQUIER, Pélissanne (FR); Yohan LE DIRAISON, CACHAN (FR); Stéphane SERFATY, Achères (FR); Pierre-Yves JOUBERT, Sceaux (FR)

(73) Assignees: UNIVERSITE PARIS-SACLAY, GIF-SUR-YVETTE (FR); CENTRE NATIONAL DE LA RECHERCHE SCIENTIFIQUE, PARIS (FR); CY CERGY PARIS UNIVERSITÉ, Cergy-Pontoise Cedex (FR)

(21) Appl. No.: 18/710,318

(22) PCT Filed: Nov. 18, 2022
(86) PCT No.: PCT/EP2022/082438

§ 371 Date: May 15, 2024

(30) Foreign Application Priority Data
Nov. 19, 2021 (FR) FR2112292

(51) Int. Cl. G01N 27/04; G01N 33/12

(52) U.S. Cl. CPC G01N 27/041; G01N 33/12

(57) ABSTRACT

A method and system for characterizing media, in particular dielectric media, using a multifrequency resonator that is positioned remote from the region to be characterized and which is excited and interrogated remotely via inductive coupling to a probe. A reader measures, via a single channel, the complex impedance of this probe when it is coupled to the resonator and when this resonator interacts magnetically with the region to be characterized. The impedance is spectrally analyzed in order to extract therefrom individual complex impedances for each of the resonance frequencies of the resonator. These individual impedances are processed in order to calculate electrical properties of the medium, such as permittivity and/or conductivity, which provide information on the nature and/or change of the medium. Also, a multifrequency resonator provided for such a system or method.

The invention relates to a method and system for characterizing media, in particular dielectric media, using a multifrequency resonator that is positioned remote from the region to be characterized and which is excited and interrogated remotely via inductive coupling to a probe. A reader measures, via a single channel, the complex impedance of this probe when it is coupled to said resonator and when this resonator interacts magnetically with the region to be characterized. The impedance is spectrally analyzed in order to extract therefrom individual complex impedances for each of the resonance frequencies of the resonator. These individual impedances are processed in order to calculate electrical properties of the medium, such as permittivity and/or conductivity, which provide information on the nature and/or change of the medium.

The invention also relates to a multifrequency resonator provided for such a system or method.

This resonator typically comprises a plurality of interrupted transmission lines, each forming a pattern that closes back in on itself and each determining a resonance frequency that is different from the others. These lines are nested inside one another or otherwise, for example single-or multiturn concentric split rings.

### PRIOR ART

In many fields in which non-destructive testing is carried out, for example for health or for quality control, it is sought to characterize a medium through its electromagnetic characteristics. This is sought, inter alia, for “dielectric” media, which are often not very conductive, and encompass in particular complex materials, organic and inorganic media, and composite materials.

This electromagnetic characterization uses in particular quantification of the interactions between the medium to be characterized and an electromagnetic field passing therethrough.

For this, it is known practice to use passive sensors, using a transmission line to which a resonant antenna is coupled. Certain sensors of this type are remotely interrogatable LC passive sensors which have a capacitive effect. This communication uses electrical resonance as a means for remote interrogation in the same way as an RFID tag, as disclosed in the publication by Huang et al.: “LC Passive Wireless Sensors Toward a Wireless Sensing Platform: Status, Prospects, and Challenges,” Journal of Microelectromechanical Systems, vol. 25, no. 5, pp. 822-841, 2016.

However, most existing characterization sensors, for example coaxial sensors, microwave antennas, or circular transmission lines (or SRRs for “split-ring resonators”) operate in contact with the medium to be tested. They furthermore operate at fairly high frequencies of about few 100 MHz to a few tens of GHz and usually only allow relative permittivity to be measured.

In the field of medical imaging, passive antennas have been developed for the non-invasive characterization of certain tissues, such as for the detection of breast cancer with microwaves (of the order of GHz) as in the publication by Modiri et al.: “Review of breast screening: Toward clinical realization of microwave imaging: Toward,” Medical Physics, vol. 44, no. 12, e446-e458, 2017; or for an imaging device such as MRI as radiofrequency antennas (of the order of MHZ) as disclosed in the publication by Gruber et al.: “RF coils: A practical guide for nonphysicists,” Journal of Magnetic Resonance Imaging, vol. 48, no. 3, pp. 590-604, 2018.

These applications use a passive antenna known as a multiturn transmission-line resonator, or “MTLR”, which has been around for about twenty years. This resonator is formed by a single interrupted transmission line, which is circular and has multiple turns, and has a given resonance frequency.

This type of resonator is described, for example, in the publication by Serfaty, N. Haziza, L. Darrasse and S. Kan, “Multiturn split-conductor transmission-line resonators,” Magnetic Resonance in Medicine, vol. 38, no. 4, pp. 687-689, 1997. These radiofrequency (“RF”) antennas are used to detect the nuclear magnetic resonance (NMR) signal emitted by the molecules of the human body when it is subjected to an intense magnetic field.

This type of multiturn transmission-line resonator (MTLR) has now been in use for a few years (at least since 2014) in measuring the dielectric properties of organic and inorganic media contactlessly via inductive coupling. This is described, for example:
for the characterization of biological tissues, in the publication by Masilamany et al. (2014): “Wireless implementation of high sensitivity radiofrequency probes for the dielectric characterization of biological tissues,” in IEEE MeMeA 2014-IEEE International Symposium on Medical Measurements and Applications, Proceedings, 2014, pp. 1-6.
for the evaluation of dielectric inclusions in a medium, in the publication from 2018: A. Pasquier, Y. Le Diraison, S. Serfaty and P. . . . Y. Joubert, “Multi-frequency detection of a dielectric object using flexible contactless RF sensors for tissue diagnosis,” 2020 42nd Annual International Conference of the IEEE Engineering in Medicine & Biology Society (EMBC), 2020, pp. 4105-4108, doi: 10.1109/EMBC44109.2020.9175769.
for the evaluation of the depth of burns in organic tissues, in the publication from 2019: Dinh, Serfaty, & Joubert-“Non-Contact Radiofrequency Inductive Sensor for the Dielectric Characterization of Burn Depth in Organic Tissues”, in Sensors, 19 (5), p. 1220;
for the evaluation of the gelation of the yogurt, in the publication of 2017: Dinh, Bore, Serfaty & Joubert-“Non invasive evaluation of yogurt formation using a contactless radiofrequency inductive technique”, in Stud. Appl. Electromagn. Mech. Electromagn. Nondestruct. Eval. Vol. 42 pp. 42 298-305 (2017).

A sensor of this type has a resonance frequency that is specific thereto, and provides a good degree of sensitivity when it is in operation and excited at its resonance frequency.

However, this type of characterization is not always accurate when it comes to obtaining certain characteristics of the environment being investigated, for example in terms of the nature and composition of the medium, or the structural characteristics thereof, in particular for complex and/or changing media.

One aim of the invention is to overcome all or some of the drawbacks of the prior art. In particular, a more accurate and/or more varied characterization of the nature (for example the composition, hygrometry, physical-chemical and biological change thereof) and/or of the structure of the medium is sought, for example by more precisely investigating the electrical properties, at different scales over time and in space; also sought is greater flexibility in the implementation thereof, for example in order to allow simpler, less invasive measurements, which are more suitable for the functional context, or more indicative of the state or change of the environment being investigated.

### PRESENTATION OF THE INVENTION

For this, the invention provides a method for characterizing at least one region to be investigated within a medium to be characterized, the method comprising at least the following steps.

A step of contactlessly inductively coupling a probe, simultaneously, to a plurality of transmission lines which are arranged so as to have different resonance frequencies from one another. These transmission lines together form a multifrequency resonator, which is located in the vicinity of said investigated region but without requiring contact with said investigated region, the transmission lines of which interact with the region to be investigated. These lines typically also interact with one another. Each of these transmission lines, or “TLRs” for “transmission-line resonators”, can thus be considered as an “elementary transmission line” or an “elementary resonator” within such a multifrequency resonator.

A step (104) of measuring the variation in the impedance of said multifrequency resonator, which can take the form of a single signal constituting a single measurement channel, by means of a reader that interacts with said probe. The reader is, for example, an external apparatus connected thereto, such as a vector network analyzer of conventional type. It can also be integrated into all or part of said probe.

A step (103) of processing said measurement of variation in impedance, comprising a spectral analysis (103) according to frequency, so as to determine a plurality of individual impedances (Zm1-Zm8) measured for a plurality of measurement frequencies (fm1-fm8, respectively). This spectral analysis is typically a vector analysis providing the modulus, the offset, the frequency, the phase, the real part and/or the imaginary part of said impedance. It is carried out, for example, over a wide band in one or more segments, so as to cover all of the expected resonance frequencies.

The measurement frequencies are defined around the resonance frequencies, in particular by means of an analytical model. Typically, the measurement frequencies are determined relative to the resonance frequencies, for example in a plurality of bands each corresponding to one of the resonance frequencies, in particular including such a resonance frequency.

A step (104) of processing one or more of said individual impedances in order to extract one or more electrical properties of said investigated region.

Typically, each of these transmission lines is formed along an “almost-closed” path, i.e. a path that closes back in on itself while leaving a non-conductive interruption, called a “gap”. This line can take various shapes and sizes while maintaining a linear path that closes back in on itself sufficiently to allow it to radiate a magnetic field.

In this way, a quantity proportional to the conductivity and/or permittivity of the medium in this region and for this frequency is obtained, for example.

Typically, these frequencies are determined by characteristics such as the dimensions and the material of the lines of the resonator, the arrangement thereof relative to one another and to any substrate, and the characteristics in terms of dimensions and nature of such a substrate.

The inventors have identified a model that makes it possible to predict the resonance frequencies of the multifrequency resonator on the basis of characteristics of this type.

Another model is used to extract the values of the complex (in the mathematical sense of the term) electrical properties from the impedance values measured by the reader.

The invention thus provides a method that implements a contactless inductive device intended for the characterization of complex dielectric media. It relates to a simple passive radiating multiresonator with a low financial and ecological cost and which is remotely interrogatable and excitable via inductive coupling. Such a device is referred to herein as a “multifrequency resonator” or WMFR for “wireless multifrequency resonator”.

It exhibits high selectivity which allows it to interact with the surrounding environment by means of radiated electromagnetic waves, the spectral spreading of which is controlled in order to contactlessly follow complex dielectric properties. In the presence of a variable electromagnetic field, typically generated by a nearby excitation coil which is referred to herein as a “probe”, this high-quality-coefficient device resonates at multiple different frequencies, which are predetermined by the sizing of the conductive tracks (typically rings) which can be viewed as interwoven radiating transmission lines.

Reciprocally, the WMFR generates, for each of these frequencies, a magnetic field, the spectral response of which is modified by the dielectric properties of the surrounding medium, referred to as the “investigated medium”. The equivalent impedance thereof around each of the resonance frequencies can then be measured via inductive coupling by transmitting/receiving using the probe.

The invention thus makes it possible to obtain more varied and/or more accurate information, in particular at the mesoscopic scale, i.e. in approximately the millimeter-to-centimeter range.

Because of its structure, such a WMFR is a passive resonator with N resonance frequencies, the N frequencies being adjustable by sizing the transmission lines. The interrogation of this resonator to access its impedance spectrum is typically carried out using a single measurement channel, simply by measuring the impedance across the terminals of the inductive circuit of the probe, for example by means of a vector network analyzer.

Since the resonator is passive and interrogatable remotely, and is additionally inexpensive and compact, it can easily be integrated into the medium under investigation, optionally permanently, for example be portable or even implantable, in particular for medical applications or for monitoring the deterioration of an object.

It can be produced on a planar and flexible support, which makes it minimally invasive. It is relatively simple and inexpensive to produce, in particular by means of printed circuit board technologies, which are flexible and tried and tested. It can therefore be integrated into numerous structures. It can also be produced using microtechnologies on biocompatible polymers for biological or medical applications.

Such a measurement technique is free of contact with the medium to be investigated, non-invasive, and multifrequency. The multifrequency resonator itself is passive and can therefore be integrated into a structure, container or packaging, for example for quality control or for monitoring the deterioration of an object. It can also be affixed to a plant, for example to monitor growth or ripening. It can also be worn as an accessory or in a clothing, or implanted into an organism in vivo, for example for a medical application or for wellbeing. It can be disposable, for example if placed in a medical dressing or in packaging for a food product. In use, the WMFR can be associated with simple and remote single-channel reader electronics while allowing spectral analysis of the dielectric properties that are sought.

Unlike conventional sensors or systems for the dielectric characterization of media (such as, for example, with microwave antennas which require electrical coupling to a transmission line or an electrical connection), such a multifrequency WMFR is passive and can be interrogated remotely in the context of a use as disclosed herein.

Since the WMFR is free of contact with the medium, the measurement is not disrupted by the quality of the contacts of the sensor with the medium being investigated, unlike in the case of capacitive sensors or bio-impedance electrodes. Furthermore, because the WMFR allows simultaneous multifrequency measurement, the associated characterization system allows a more robust estimate of the dielectric properties that are sought.

In addition to the fact that access to the dielectric parameters of the medium at multiple frequencies enriches the quality of the characterization, it also makes it possible, where appropriate, to use one or more analysis frequencies to calibrate the WMFR with respect to influence quantities that are potentially troublesome for the measurement, for example large variations in temperature, variations in distance between the WMFR and the medium to be investigated, or variations in the volume of the measurand.

The variation in geometry associated with the various resonant elements of the WMFR makes it possible to envisage a device for which the regions of investigation, in particular in terms of depth, are directly dependent on the characteristics of the element that is brought into resonance.

Specifically, for resonant antennas, the depth of penetration of the generated magnetic field into the medium is directly related to the diameter thereof. Here, since the various resonant elements (the various transmission lines) of the WMFR have different dimensions from one another, for example by way of a nested structure, each thereof will have a different inspection depth, which gives the investigation a tomographic character. This can prove to be particularly advantageous in many cases, for example in the context of detecting and/or locating dielectric anomalies in a medium, such as a tumor in a tissue, or a crack in concrete or steel.

By way of comparison, methods such as coaxial sensors and capacitive sensors require contact with the medium; radiofrequency and microwave antennas further require an electrical connection for use thereof. It should also be noted that most of these sensors, in particular coaxial sensors and microwave antennas, operate at frequencies from a few 100 MHz to a few tens of GHz, i.e. higher than those of the method disclosed herein. They therefore access dielectric parameters of the medium that are essentially related to the relative permittivity of the medium, but only with difficulty allow the electrical conductivity of media to be measured.

Many sectors can make use of the invention. These include, for example and in a non-limiting manner, the following sectors:

Agri-food sector: characterizing plants and monitoring the health of animals; monitoring food processes and, for example, the growth, ripening, hydrometry and aging, and controlling the freshness and quality of food, in particular during storage, transport, packaging, and natural, or accelerated or artificial, ripening Health sector, by characterizing biological tissues via the dielectric properties thereof: detecting lesions such as tumors or other internal lesions, characterizing lesions, such as for tumors, injuries, burns or skin diseases, and monitoring the change thereof over time.

Characterizing different types of soft materials, or geophysical materials for characterizing soils, cosmetics, etc.

Integrated health monitoring (or structural health monitoring) for different types of objects or works: civil engineering, aerospace; more particularly for all poorly conductive media such as carbon, concrete, wood, composite materials.

Many of these applications or others can then take the form of connected objects, participating in or providing such functions.

Various specific features of the invention will be explained, with these features being able to be combined with one another or taken individually.

According to one specific feature of the invention, the individual impedances of multiple different resonance frequencies, or the respective electrical properties extracted therefrom, are combined to provide a characterization of the region to be investigated in portions that are located at different distances from the multifrequency resonator. This makes it possible, for example, to provide imaging and/or tomography of the region to be investigated.

In particular, the size of the antenna has a direct effect on penetration depth, and therefore the position of the investigated region in terms of depth.

According to another specific feature, the individual impedances of multiple different resonance frequencies, or the respective electrical properties extracted therefrom, are combined to provide a more accurate characterization of the same portion of the region to be investigated, for example by using the values derived from the different frequencies to provide a more accurate value of an electrical property or, for example, by using the different frequencies to extract different properties which apply to the same investigated region and can therefore give more accurate information on said region.

According to yet another specific feature, this method is used to produce a plurality of characterizations at different times, so as to provide monitoring over time of a region to be investigated including at least one material or object undergoing change.

According to yet another specific feature, this method implements one or more multifrequency resonators which are integrated or implanted into an object or system so as to characterize a region to be investigated, which region to be investigated includes a material belonging to said object, and/or a material in contact with or in the vicinity of said object, and/or an interface between said materials. According to this specific feature, this method is implemented so as to obtain a plurality of time-distributed characterizations for said region to be investigated and thereby provide monitoring over time of a change in the region to be investigated.

This makes it possible, for example, to monitor the condition of a material, such as its leaktightness, or the appearance of new materials or phenomena, or to monitor the static or dynamic properties of a fluid or solid body located or passing in the vicinity of or in contact with said object.

### System and Device Implementing the Method

According to another aspect of the invention, a system for contactlessly characterizing at least one region, referred to as an investigated region, within a medium to be characterized is provided, the system comprising:
at least one transmission-line resonator (in the linear but not necessarily straight sense), which is intended to be arranged in the vicinity of said investigated region, but without requiring contact with said investigated region,
a probe arranged so as to:
   on the one hand, be coupled via inductive coupling to said resonator by means of an inductive loop circuit, and
   on the other hand, to interact with at least one reader (typically via electrical connection; the reader can be integrated into the same housing as said probe or otherwise);

According to the invention, this resonator comprises a plurality of transmission lines which are arranged to have different resonance frequencies from one another and thereby form a multifrequency resonator, the transmission lines of which interact with the region to be investigated, and typically also with one another. Typically, these interactions bring about changes in the individual responses of said transmission lines, in particular in terms of resonance frequency and bandwidth around this resonance frequency.

This same probe is arranged so as to interact simultaneously with all of said multifrequency resonator, i.e. with the assembly formed by the plurality of lines. This reader is then arranged so as to interact with said probe in such a way as to implement a method as disclosed herein. The electrical properties provided by the method are then used to characterize the medium in the region to be investigated.

Typically, this elementary transmission line is formed by conductive tracks deposited on the two opposite faces of a low-loss planar insulating substrate. These conductive tracks are preferably arranged relative to one another so that the one or more interruptions therein on one face are not opposite the one or more gaps on the other face. It is however also possible to use just one track for each transmission line.

The system according to the preceding claim, characterized in that the multifrequency resonator comprises a plurality of transmission lines which are separate and not connected to one another, and which are each formed by at least one conductive track produced on a two-dimensional dielectric substrate, which is typically poorly or not conductive, possibly planar or non-planar in shape, and, for example, flexible. This track is formed along an “almost-closed” path, i.e. a path that closes back in on itself while leaving a non-conductive interruption. This track can take various shapes and sizes while maintaining a linear path that closes back in on itself sufficiently to allow it to radiate a magnetic field. This type of transmission line is, for example, a track in the form known as a circular “split ring”. Other patterns are envisaged: circular or otherwise, rounded or otherwise, and possibly in a spiral or of variable radius.

Preferably, each line comprises two tracks each formed on one of the two opposing faces of the same substrate, facing and angularly opposite one another.

Within the plurality of transmission lines, the different resonance frequencies are typically obtained by giving them different dimensions. Alternatively or in combination, these differences in frequency are obtained through different combinations of one or more parameters selected from among the length of the track, the geometry of the path thereof (typically the radius), the width of the track, the thickness of the track, the nature of the material of the track, the thickness of the substrate, and the nature of the material of the substrate.

Provision is also made to produce an elementary transmission line with one or more openings, i.e. formed by multiple tracks which are not connected to one another but are sufficiently close to and/or interwoven with one another to radiate at the same frequency and in the same spatial region.

Provision is also made to produce an elementary transmission line with more than two layers, or with a single layer.

According to one specific feature, all or some of the transmission lines of the multifrequency resonator each form an almost-closed path with a single turn, typically a single-turn circular split ring.

Alternatively or in combination, provision is also made to use multiturn paths, for example in a continuous spiral or with discrete offsets, and in particular in a known manner.

According to another specific feature, the multifrequency resonator comprises a plurality of conductive tracks of almost-closed shape which are enclosed inside one another.

According to yet another specific feature, the multifrequency resonator comprises a plurality of transmission lines which are separate and not connected to one another, and which are each formed by a conductive track produced on a two-dimensional insulating substrate, which are in particular paired with one another on both sides of said substrate, and facing and angularly offset, along a “quasi-closed” and in particular circular path. According to this specific feature, at least two transmission lines of different frequencies are arranged such that they are not nested inside one another, and are referred to as separate. The probe then comprises an inductive loop circuit, the shape of which is arranged so as to be able to obtain simultaneous inductive coupling to said two separate transmission lines, forming in particular a conductive loop that has single-turn or multiturn projections that are arranged so as to be able to position a projection over each of said separate transmission lines. It can, for example, be in a daisy pattern, where the different petals thereof each cover one of the split rings of a resonator with a plurality of separate split rings.

According to yet another specific feature, the multifrequency resonator comprises a plurality of transmission lines which are separate and not connected to one another, and which are formed by conductive tracks (c14a, c14b) each produced along a “almost-closed”, and in particular circular, path, in a coplanar manner or on the same two-dimensional insulating substrate.

All or some of said lines can also be arranged in a non-coplanar manner with respect to one another.

They are, for example, arranged on separate and substantially parallel planes (or two-dimensional surfaces). These planes or surfaces are, for example, strictly parallel to one another, or form an angle of less than 45°, in particular of less than 20° or even of less than 5°, with respect to one another.

Each line can also have a three-dimensional path, i.e. a path that is not inscribed within a simple two-dimensional surface, provided that this path allows a directed magnetic field to be radiated with flux lines that pass in common through the various transmission lines. It can, for example, take the form of a spiral arrangement on a cone, of a circular ring around an axis but with variations in axial position along its path.

According to another example, they are arranged on two-dimensional planes or surfaces that are not parallel to one another, for example in order to be coupled in different directions to different inductive loops or portions of the probe.

According to one preferred embodiment, this multifrequency resonator comprises a plurality of transmission lines which are separate and not connected to one another, and which are each formed by a conductive track produced on the same two-dimensional insulating substrate, each along an “almost-closed” path, for example a single-turn and in particular circular path, wherein these transmission lines are arranged inside one another, and in particular concentrically with respect to one another.

According to yet another aspect of the invention, a transmission-line resonator device is provided, this device being of the type capable of communicating via inductive coupling with an inductive loop probe in order to be excited by said probe, so as to interact with said probe. According to this aspect, this resonator comprises a plurality of transmission lines of almost-closed shape which are enclosed inside one another, in particular concentric circles, and which are arranged so as to form, together, a multifrequency resonator suitable for being implemented within a system or a method as disclosed herein.

According to one specific feature, each transmission line forms a single turn.

### Implanted Resonator(s)

According to another aspect of the invention, an object or system is provided, this object or system comprising at least one transmission-line resonator, which resonator comprises a plurality of transmission lines which are arranged to have different resonance frequencies from one another and thereby form a multifrequency resonator, the transmission lines of which interact with the region to be investigated, and typically also with one another.

According to this aspect, this multifrequency resonator is arranged so as to form an inductive coupling with an inductive loop probe, so as to interact with said probe:
to form therewith a characterization system as disclosed herein, which system is arranged so as to characterize a region to be investigated (possibly remote from said resonator) within a medium to be characterized, and/or
to be implemented within a method as disclosed herein;
   Typically, this region to be investigated includes a material belonging to said object, and/or a material in contact with, or in the vicinity of said object, and/or an interface between said materials.

Thus an “implanted object” or an “implanted system” is obtained which can be monitored in a regular or even continuous manner, by a separate or integrated reader, for example in a number of locations or in locations that are difficult to access, and in a very regular manner since the one or more resonators remain in a very stable position relative to the object or system thus implanted.

According to one specific feature, this object or system comprises at least one probe arranged so as to be able to communicate with the multifrequency resonator via inductive coupling, wherein said multifrequency resonator and said probe are arranged so as to form a characterization system as disclosed herein or to be implemented within a characterization method as disclosed herein.

Various embodiments of the invention are provided, incorporating, according to all of their possible combinations, the different optional features set out herein.

### BRIEF DESCRIPTION OF THE DRAWINGS

Other features and advantages shall become evident from the detailed description of an entirely non-limiting embodiment, and from the enclosed drawings in which:

FIG. 1a and FIG. 1b are scale top views showing multifrequency resonators according to exemplary embodiments of the invention, where:

FIG. 1a: shows a resonator with four nested single-turn circular meshes, and

FIG. 1b: shows a resonator with eight nested single-turn circular meshes

FIG. 2 is a scale top view showing a multifrequency resonator according to one exemplary embodiment of the invention, with four nested circular meshes, one of which is single-turn and the other of which is multiturn;

FIG. 3a and FIG. 3b show an example of a single-turn circular elementary transmission line (or TLR), which can be used within the multifrequency resonator according to one exemplary embodiment, where:

FIG. 3a: a partial schematic top view, cross-sectional view and exploded perspective view,

FIG. 3b: a circuit diagram of the interactions between the TLR of FIG. 3a taken individually, an inductive control loop, and a vector network analyzer;

FIG. 4 is a partial schematic top view and cross-sectional view of a multigap elementary transmission line (or TLR) with six circular transmission lines in portions and with decreasing radii, which are concentric with respect to one another and are offset by 60°, each formed by two tracks which are each arranged on one of the two faces of an insulating thin substrate, and facing one another and angularly offset with respect to one another;

FIG. 5 is a scale perspective view of a test bench using the resonator of FIG. 1b with eight resonance frequencies to characterize the liquid content of an insulating container, via interrogation through the bottom wall of the container,

FIG. 6 is an equivalent circuit diagram of a resonator with three resonance frequencies, showing the interactions thereof with a probe and with a medium to be characterized;

FIG. 7 is a curve showing, for the eight-resonance resonator of FIG. 1b, the modulus of the measured impedance Zmes with frequency, overall and with a close-up on the low-intensity peaks;

FIG. 8 is a curve showing, in a simulated manner, for the four-resonance resonator of FIG. 1a with no load, the real part of the measured impedance Zmes with frequency;

FIG. 9 is a curve similar to that of FIG. 8, simulated for the eight-resonance resonator of FIG. 1b;

FIG. 10 is a graph showing a comparison of the resonance frequencies as obtained: experimentally, modeled semi-analytically, and by simulation:
(a): for the four-frequency resonator of FIG. 1a, and
(b): for the eight-frequency resonator of FIG. 1b;

FIG. 11 is a graph individually showing, for each of the four meshes of the four-frequency resonator of FIG. 1a, the resistance induced according to the conductivity of the medium to be characterized, formed by an aqueous solution of NaCl;

FIG. 12 is a flowchart schematically showing the operation of the characterization method with a number of exemplary embodiments of the invention;

FIG. 13 is a top-view diagram showing a multifrequency resonator with four frequencies, interrogatable by the same probe, according to one exemplary embodiment of the invention in which the resonator comprises two meshes nested within one another and two other, non-nested meshes;

FIG. 14 is a top-view diagram showing a multifrequency resonator, interrogatable by the same probe, according to one exemplary embodiment of the invention in which the resonator comprises three separate groups, each comprising four transmission lines nested within one another;

FIG. 15 is a cross-sectional diagram showing one exemplary embodiment of the invention for contactlessly characterizing the content of a container that is permeable to the magnetic field;

FIG. 16 is a cross-sectional diagram showing one exemplary embodiment of the invention for contactlessly characterizing the content of a container comprising a resonator implanted in the thickness of the wall thereof;

FIG. 17 is a cross-sectional diagram showing one exemplary embodiment of the invention for contactlessly characterizing the condition of the inner surface of a tank comprising a plurality of resonators implanted in the wall thereof;

FIG. 18 is a diagram showing, according to one exemplary embodiment of the invention, a use of a matrix device comprising a plurality of multifrequency resonators, each with its own individual probe, to characterize graphically, and with respect to change over time, an organism in vivo;

FIG. 19 is a cross-sectional diagram showing, according to one exemplary embodiment of the invention, a use of a device comprising a multifrequency resonator and its own individual probe to monitor the change over time in a biological body undergoing decomposition. (a veal steak in this instance)

FIG. 20 is a graph showing the inductance induced in the body of FIG. 19 over time;

FIG. 21 is a graph showing the resistance induced in the body of FIG. 19 over time.

### DETAILED DESCRIPTION AND EXEMPLARY EMBODIMENTS WITH REFERENCE TO THE FIGURES

The WMFR itself is a passive structure consisting of the association of interrupted-transmission-line resonant passive structures, which can be represented by the same number of coupled RLC circuits as transmission lines thus implemented. Each of these individual transmission lines is referred to herein as a TLR, for “transmission-line resonator”, or “mesh” since it is equivalent to a circuit mesh in an equivalent circuit diagram such as that of FIG. 6.

FIG. 1a and FIG. 1b show examples of multifrequency resonators according to exemplary embodiments of the invention. FIG. 1a shows one example of a multifrequency resonator MR1 with four lines M11 to M14, which are nested concentric circular single-turn lines. FIG. 1b shows one example of a multifrequency resonator MR2 with eight lines M21 to M28, which are nested concentric circular single-turn lines.

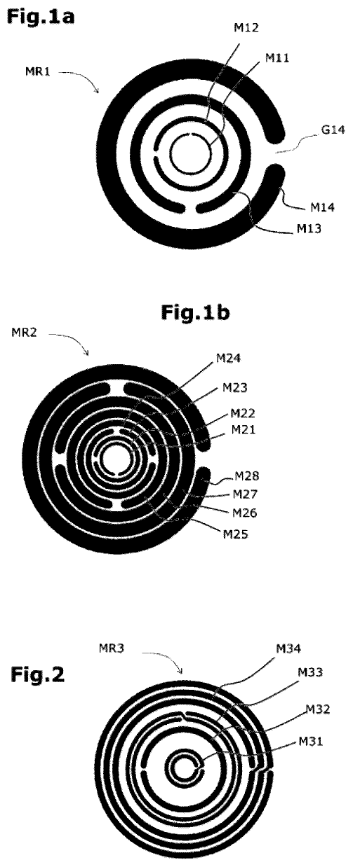

FIG. 2 shows another example of a multifrequency resonator MR3, with four nested concentric circular lines M31 to M34, one M32 of which is single-turn and the others M31, M33, M34 of which are multiturn.

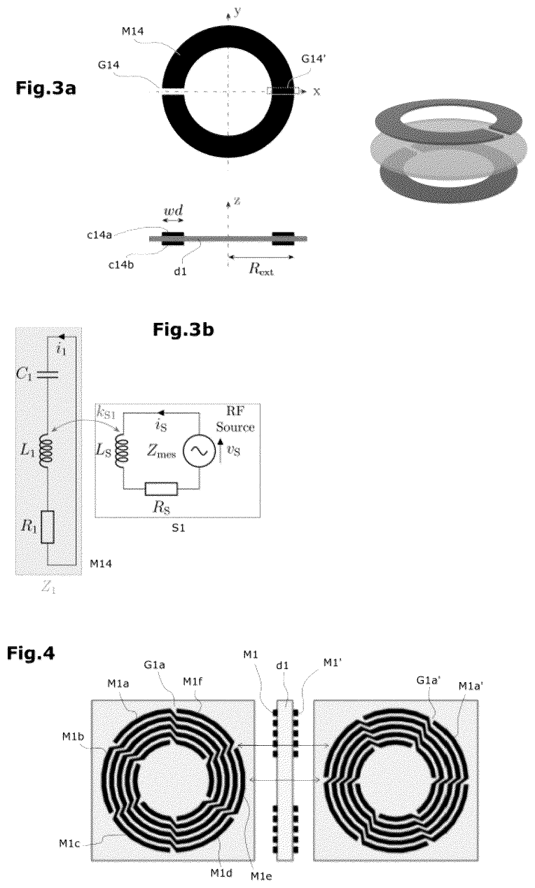

FIG. 3a shows the three-dimensional arrangement of one of the transmission lines of such a resonator, in this case the largest single-turn line M14 of the resonator of FIG. 1a. As can be seen in the cross-sectional view, this “transmission line”, in this instance considered to be a single element, is formed here by two interrupted (or “open”) circular tracks, with one circular track arranged on each of the two faces of a thin dielectric or insulating substrate, in this instance a flexible Kapton substrate. Each open track within a line has an interruption or space, referred to herein as a “gap”, for example referenced G14 for the line.

It is possible to produce this transmission line as a single track, but the arrangement in a group of multiple tracks of the same size placed facing one another is preferred, inter alia for improved effectiveness.

In the embodiments with two (or more) tracks, they are preferably planar and parallel to one another, and/or radially identical. This arrangement is ensured here by positioning them on both faces of the substrate, but any of the technical solutions that allow this arrangement to be obtained can also be used.

Typically, the various tracks forming the same transmission line are arranged facing one another, with their respective gaps offset angularly with respect to one another. In this example, the gap G14 of the upper track (this upper track here being referenced M14 like the transmission line in which it participates) is situated facing a solid portion of the opposite track M14′. Likewise, the gap G14′ of the lower track M14′ is arranged facing a solid portion of the upper track M14. Preferably, their respective gaps are each in an angular position that is as far as possible from the one or more gaps in the opposite track, in this case at diametrically opposite locations.

Unlike conventional resonators, which instead seek to minimize the common mode current, maximization of the common mode current is thus obtained, since the offset between the two faces allows differential-mode compensation.

These two tracks will together form a single transmission line M14, which has a single resonance frequency, which is determined in particular by the geometry of these tracks.

FIG. 4 shows another example, referred to as multi-opening or “multigap”, of a transmission line M1 operating with a single resonance frequency. Each face of the substrate d1 bears a track M1 (and M1′), which consists of multiple portions which are not connected to one another. In this example, there are six portions M1a to M1f, which are circular in portions and with decreasing radii, and are concentric with respect to one another and are offset by 60°. These portions are each formed by two tracks each arranged on one of the two faces of an insulating thin substrate, facing and angularly offset with respect to one another. Each track has a gap, for example G1a for the track M1a. On the opposite face of the substrate, the associated multiportion track M1′ has the same arrangement of six portions, but the gaps thereof are angularly offset relative to those of the multiportion track M1. Thus, the gap G1a′ of the portion Ma′ of the face shown on the right is offset by 30° relative to that, G1a, of the other face. Each of these gaps G1a and G1a′ therefore faces a solid portion of the opposite track. Although this design has multiple track portions which are not connected to one another, the nested arrangement thereof allows them to operate together as a single transmission line with a single resonance frequency. This multiportion, or multi-opening, line can also be used as one of the lines of a multifrequency resonator, instead of a single-portion line, and in any of the possible combinations thereof.

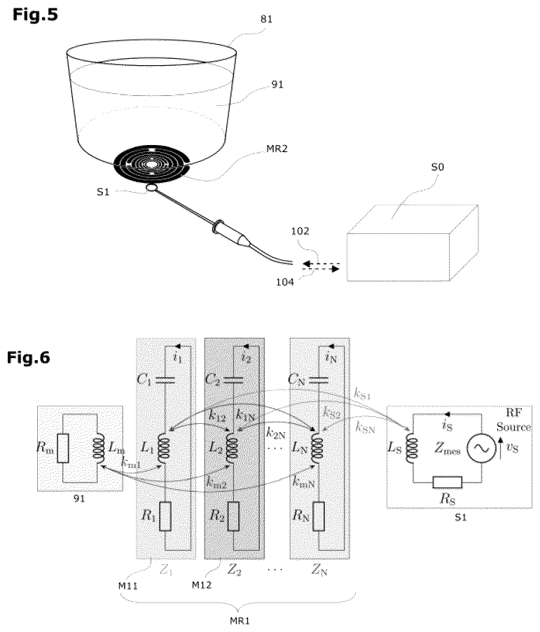

FIG. 6 shows a circuit diagram of interactions which shows the implementation of this type of structure in an experimental setup combining a remote excitation/reading coil connected to a vector analyzer on the one hand, and placed in the vicinity of a medium to be investigated on the other hand (see FIG. 5 and FIG. 15 to FIG. 19).

This analytical model was validated experimentally and by finite element numerical modeling from HFSS. By virtue of this complete model, it is possible to size the WMFR and thus choose the desired spectral response. It is also possible to extract the impedance induced in the environment surrounding the WMFR resonator, which is representative of complex dielectric properties. These models were confirmed by test bench with an accuracy of around a few %.

The step of extracting the induced impedance of the medium around each resonance frequency is done using a complex fitting algorithm for fitting between the measurement data and the interaction model derived from the diagram of FIG. 3a. By way of example, the conductivity spectrum of an organic medium (veal steak) that was left for five days at room temperature is shown, measured using a WMFR with eight resonances between 20 MHz and 350 MHz (FIG. 5). The conductivity with frequency of observation and time is observed.

### Modeling and Resonance of the Elementary Transmission Line (TLR)

The resonance condition of this elementary transmission line, or TLR, can be determined from a semi-analytical model involving the constituent and geometric elements of the resonator.

For example, in the case of a circular and single-turn transmission line (TLR) as in FIG. 3a, the following constituent and geometric elements are used:
outer radius: Rₑₓₜ
track width: W_{d}
conductor thickness: th_{cond}
dielectric thickness: th_{dielec}

In the case of a circular and multiturn transmission line, such as the lines M31, M33 and M34 of the resonator MR3 of FIG. 2, other parameters must also be taken into account, such as:
number of turns: Nₜ
distance between the tracks
the number of openings (gaps): n

The set of these parameters contributes to the resonance condition of the elementary transmission line, which is written according to the following equation:

$$\frac{Λ \cdot ω}{4 n \cdot Z} \cdot tan ( \frac{ω \cdot \sqrt{{ϵ}_{re}} \cdot {x}_{l}}{4 n \cdot c} ) = 1$$

where:
“ω” is the angular frequency
“n” is the number of openings
“∧” is the equivalent total inductance of the resonator
“Z” is the characteristic impedance of the transmission line
“Eᵣₑ” is the effective relative permittivity of the line
“xᵢ” is the mean line length
“c” is the speed of light in vacuum

Based on this relationship, and the work undertaken in documents [22], [23], [24], and [25](listed in the annex) which make it possible to determine the couplings between transmission lines and the characteristic impedance thereof, it is possible to predetermine the resonance frequency of the TLR, the total inductance Lₑₜ thereof and the equivalent capacitance C thereof. Such a semi-analytical model, or SAM, was written in the Matlab environment in the present case.

Based on this, it is possible to complete the equivalent RLC circuit diagram for this elementary resonator by estimating the equivalent resistance of the whole, R, based on considerations regarding the geometry of the tracks, in particular the conductors used, and the surface state of the tracks, as the inventors have shown in the document (document described in the annex). However, determining the latter remains rather empirical.

In order to fully characterize the elementary resonator, in order to obtain better accuracy for the determination of R, it is proposed here to deduce the equivalent resistance R from a measurement of the resonance quality factor Q thereof.

The implementation of such a TLR (single-line resonator) as resonant inductive sensor has been described and published by the inventors in applications [12], [26] and [27](documents described in the annex). The implementation consists in exciting the resonator via inductive coupling using a remote control loop, which is itself connected to a vector network analyzer (VNA, for example an HP4195A analyzer), as shown in FIG. 5.

The VNA delivers a radiofrequency signal that supplies the exciter loop with power. The magnetic flux from this loop induces, via magnetic coupling, a current in the tracks of the TLR placed in the vicinity, the intensity of which increases the closer it is to the resonance of the TLR. Reciprocally, the TLR generates a magnetic field which will “modify” the operation of the loop and induce a variation in the impedance thereof. Since the loop is connected to the VNA, it is possible to measure the impedance thereof around the resonance frequency of the TLR. From this analysis, it is then possible to extract, via contactless inductive coupling, information on the magnetic interactions between the TLR and the immediate environment thereof. This loop, by virtue of its simultaneous exciting and measuring, can be considered a control probe that excites and “reads” the TLR. The idea is therefore to have as much power as possible transmitted by the probe without the coupling between the probe and the TLR interfering with the operation of the TLR, as explained by [26]. The probe is therefore sized so as to work below its own self-resonance frequency (and remain within a range of frequencies in which it is purely inductive); the diameter thereof is smaller than the inner diameter of the TLR with which it interacts, and it is placed at a distance close to the TLR in order to ensure sufficient coupling with the TLR. All of the interactions taking place between the TLR, the control probe and the VNA are summarized in the equivalent circuit diagram of FIG. 4.

This diagram in FIG. 4 shows two magnetically coupled electrical meshes. One of the meshes, Z₁, corresponds to the TLR which is modeled by the resonant circuit with distributed constants L₁, C₁, and R₁. It is inductively coupled to the probe represented by a circuit Lₛ and Rₛ, of total impedance Zs, the elements of which can be determined experimentally and written as:

$${Z}_{S} = {R}_{S} + j 2 π fL$$

$${Z}_{S} = {R}_{S} + j 2 π {fL}_{S}$$

The current in the branch associated with the resonator is denoted by i₁ and the voltage across its terminals by v₁. For the branch corresponding to the probe, they are denoted by is and Vs, respectively. The coefficient of coupling between the two circuits is denoted by kₛ₁. The probe is connected to the RF source of the VNA, which “sees” the impedance Zₘₑₛ across its terminals. This can readily be explained from the different elements in the diagram of FIG. 4 according to the following equation, according to [26]:

$${Z}_{mes} = {Z}_{S} + \frac{{k}_{S 1}^{2} {L}_{S} {{L}_{1} ( 2 π f )}^{2}}{j 2 π {fL}_{1} + \frac{1}{j 2 π f {C}_{1}} + {R}_{1}} \qquad ( 1.2 \text{.2} \text{.1} )$$

This expression makes it possible to predict the behavior of the association of the TLR with the probe according to the frequency used.

The interactions between the exciter probe and the TLR can also be modeled using numerical calculations, which was carried out using the finite element numerical modeling software distributed under the name “HFSS” by Ansys. These simulations also make it possible to determine, via this calculation of the frequency response of the system, the resonance frequency of the TLR with a given set of geometry and parameters.

Experimental measurements were carried out with two batches of six single-turn and single-opening TLRs, produced as a two-layer printed circuit board (PCB) on a flexible Kapton substrate, and varnished. The substrate has a relative permittivity ϵᵣ=3.4, a thickness th_{diel}=50 μm, and the conductor used for the transmission line is copper with a thickness th_{cond}=35 μm.

The various TLRs of the two batches differ from one another in different track widths and outer radii.

For each of the twelve TLRs considered, the inventors determined the theoretical resonance frequency expected using the analytical model mentioned above (SAM). In addition, they measured the resonance frequency of the various TLRs produced. Two series of measurements were carried out. For batch 1, the probe is a loop made of copper wire with a diameter of 11 mm and a circular cross section of 1.5 mm, placed at a distance of 5 mm from the TLR. For the elementary resonators of batch 2, the loop is of the same type but with a diameter of 14 mm, while maintaining the same distance between the loop and the TLR.

The four TLRs of batch 1 have the following characteristics:

TABLE 1

| | | | | | | | de- | de- |
|---|---|---|---|---|---|---|---|---|
| | | | | | | | via- | via- |
| | | | | | | | tion | tion |
| | Rₑₓₜ | w_{d} | | fₘₑₛ | f_{HFSS} | f_{SAM} | HFSS | SAM |
| Name | [mm] | [mm] | Qₘₑₛ | [MHz] | [MHz] | [MHz] | [%] | [%] |
| M11 | 10.5 | 1 | 301 | 241 | 205 | 230 | 14.9 | 4.8 |
| M12 | 18.75 | 2.5 | 385 | 92.3 | 77.7 | 93.9 | 15.8 | 2.5 |
| M13 | 30 | 5 | 489 | 44.0 | 39.8 | 43.5 | 9.5 | 2.2 |
| M14 | 47.5 | 10 | 705 | 21.2 | 19.9 | 21.1 | 6.1 | 6.5 |

The eight TLRs of batch 2 have the following characteristics:

TABLE 2

| | | | | | | | de- | de- |
|---|---|---|---|---|---|---|---|---|
| | | | | | | | via- | via- |
| | | | | | | | tion | tion |
| | Rₑₓₜ | w_{d} | | fₘₑₛ | f_{HFSS} | f_{SAM} | HFSS | SAM |
| Name | [mm] | [mm] | Qₘₑₛ | [MHz] | [MHz] | [MHz] | [%] | [%] |
| M21 | 8 | 1.5 | 303 | 295 | 281 | 302 | 5.5 | 4.7 |
| M22 | 11 | 2 | 373 | 192 | 169 | 192 | 12 | 0.93 |
| M23 | 14.5 | 2.5 | 392 | 130 | 118 | 130 | 9.4 | 0.7 |
| M24 | 18.5 | 3 | 413 | 91.7 | 82.5 | 91.7 | 10 | 0.93 |
| M25 | 23.5 | 4 | 445 | 62.9 | 59.5 | 63.7 | 5.4 | 1.2 |
| M26 | 29.5 | 5 | 482 | 45.1 | 39.6 | 45.4 | 12 | 2.6 |
| M27 | 36.5 | 6 | 504 | 33.3 | 30.6 | 33.2 | 7.9 | 5.4 |
| M28 | 44.5 | 7 | 523 | 24.9 | 22.4 | 24.9 | 10 | 3.8 |

A numerical simulation of the TLRs, using HFSS, was also carried out, which overall showed very good agreement between the expected theoretical resonance frequency (from SAM) and measurement. Indeed, for both batches, the average deviation observed is less than 6%.

Lastly, a numerical simulation was carried out, using HFSS, of the TLRs excited by probes with the same size and distance parameters as for the experimental tests. The calculated impedance spectra made it possible to extract the resonance frequency of the TLR & probe sets under consideration. Here again, a very good fit between the results of the HFSS simulation and the experimental measurements was observed, with an average deviation of less than 10%.

In conclusion, regarding the elementary resonator, comparisons were made between the TLR models and the experimental measurements. They showed that both of the models used (semi-analytical, “SAM”, and numerical, “HFSS”) are relevant in studying the behavior of TLRs, in particular in frequency ranges from 20 MHz to 300 MHz. In addition, although the semi-analytical model used does not take into account the presence of the excitation coil in calculating the resonance frequency of the TLR, the determination of the latter remains very close to that observed experimentally. This indicates that the excitation coil has only a small effect on the properties of the TLR, which from a practical point of view constitutes an advantage in terms of simplification.

### Multifrequency Contactless Resonator (WMFR)

In the examples presented herein, the contactless resonator (WMFR) is formed of multiple elementary resonators, or TLRs, which have a common substrate. This WMFR consists of conductive open tracks, made of for example copper, which are deposited on either side of a low-dielectric-loss substrate (for example Kapton, or FR4) on the two opposite faces thereof. The organization of these tracks (length, spacing, thickness, type of conductor, gap width) with respect to one another and the type of substrate used (losses, thickness) determine the various resonance frequencies associated with the various constituent elements of the WMFR, which can range from a few MHz to several hundred MHz depending on the geometries chosen, thereby covering a very broad spectrum of frequencies in the RF range. Many geometric arrangements of the conductive tracks of the WMFR are therefore conceivable, and therefore offer a wide variety of levels of performance suitable for different applications. The position of the gaps in the various coupled transmission lines making up the WMFR has no effect on the resonance frequencies observed. Optionally, it is preferable to distribute the gaps uniformly so as to make the magnetic flux generated by the WMFR uniform.

The WMFR is based on the association of multiple elementary TLRs, all excited by the same probe. When a number “N” of TLRs thus arranged are excited by the probe, all of the magnetic couplings involved can be represented using the equivalent circuit diagram of FIG. 5.

In this equivalent diagram, each of the N elementary TLRs is magnetically coupled to the excitation coil via a coupling coefficient kₛⱼ (with j∈{1, 2, . . . , N}), and to each of the other TLRs via the coupling coefficient kₘₙ (with m≠n and (m,n) ∈{1, 2, . . . , N}). In each of the branches, the currents flowing therethrough are denoted by ij and the voltage across the terminals of each is denoted by vⱼ (with j∈{1, 2, . . . , N}). The interactions between the N+1 meshes of FIG. 5 can be written in the form of a matrix system inspired by publication [15]:

$$[ \begin{matrix}{Z}_{S} & j ω {M}_{S 1} & … & j ω {M}_{SN} \\ j ω {M}_{1 S} & {Z}_{1} & … & ⋮ \\ ⋮ & ⋮ & ⋱ & ⋮ \\ j ω {M}_{NS} & j ω {M}_{N 1} & … & {Z}_{N}\end{matrix} ] [ \begin{matrix}{i}_{s} \\ {i}_{1} \\ ⋮ \\ ⋮ \\ {i}_{N}\end{matrix} ] = [ \begin{matrix}{v}_{s} \\ 0 \\ ⋮ \\ ⋮ \\ 0\end{matrix} ] \qquad ( 1.3 \text{.2} \text{.1} )$$

with M_{ab}=k_{ab} \sqrt{LₐL_{b}} for a≠b and

$${Z}_{j} = {R}_{j} + j 2 π {fL}_{j} + \frac{1}{j 2 π {fC}_{j}}$$

The equivalent electrical parameters Lⱼ and Cⱼ of the TLRⱼ are calculated analytically as above via the semi-analytical model SAM, and the resistances Rⱼ are estimated from the measurement of the quality coefficient Qⱼ of each elementary TLR taken individually. The mutuals, M_{ab} with (a,b) ∈{1, 2, . . . , N}, between two resonators are determined using the analytical formulas in publication and the mutuals, Mₛⱼ, between the probe and the resonators are determined using publication [24]. Since the elements of the matrix system of equation (1.3.2.1) have thus all been determined, it is possible to invert the system to obtain:

$$[ \begin{matrix}{i}_{s} \\ {i}_{1} \\ ⋮ \\ ⋮ \\ {i}_{N}\end{matrix} ] = [ \begin{matrix}{y}_{11} & … & … & {y}_{1 N} \\ ⋮ & ⋱ & … & ⋮ \\ ⋮ & ⋮ & ⋱ & ⋮ \\ {y}_{N 1} & … & … & {y}_{NN}\end{matrix} ] [ \begin{matrix}{v}_{s} \\ 0 \\ ⋮ \\ ⋮ \\ 0\end{matrix} ] \qquad ( 1.3 \text{.2} \text{.2} )$$

wherein the elements yij are the coefficients of the inverse matrix, which are homogeneous with admittances. The impedance Zₘₑₛ can then be extracted from equation (1.3.2.2), according to:

$${Z}_{mes} = \frac{{v}_{s}}{{i}_{s}} = \frac{1}{{y}_{11}} \qquad ( 1.3 \text{.2} \text{.3} )$$

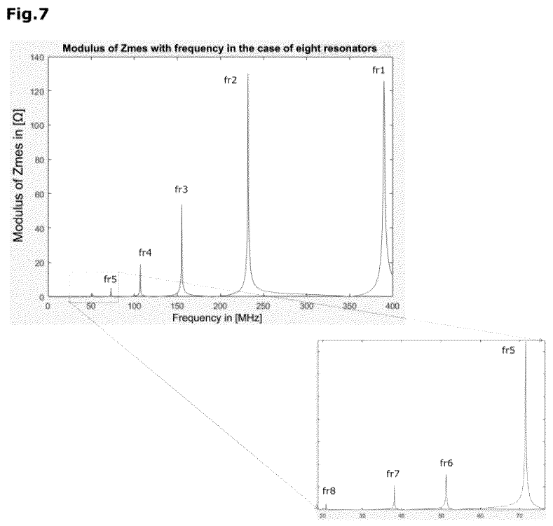

By way of example, FIG. 7 shows the numerical result, as determined analytically using equation 1.3.2.3, of determining the impedance Zₘₑₛ seen from the excitation coil, for a WMFR consisting of the association of the eight TLRs of batch 2, which form the eight-line multifrequency resonator MR2 (or “WMFR-8”) of FIG. 1b.

FIG. 7 thus shows the modulus of Zₘₑₛ in a range of frequencies from a few MHz to 400 MHz. The presence of eight resonance peaks is observed, at eight resonance frequencies fr1 to fr8, which reflects the contribution of the eight TLRs making up the WMFR-8.

### Coexistence of the TLRs Inside the WMFR

The HFSS simulation was used to study the way in which the elementary TLRs enter into resonance within the WMFR. For this, the surface distribution of the currents in the vicinity of the resonance frequencies of the various TLRs was calculated. In the case of the multifrequency resonator MR1 (or WMFR-4) of FIG. 1a, the current densities were calculated for the four working frequencies of 20 MHZ, 45 MHZ, 90 MHz and 206 MHz. For each of these selected working frequencies, it can be seen that it is the TLRs for which the theoretical resonance frequency is closest to the working frequency which are traversed by the most intense current. This simulation therefore clearly shows that each TLR making up the WMFR is indeed “actuated” selectively by the excitation coil when the latter is working at the correct tuning frequency (which is a frequency close to but different from its own resonance frequency).

This confirms that it is possible for multiple different TLRs to coexist within the WMFR without the resonance of each thereof being jeopardized, despite the couplings present between the elementary TLRs.

### Complex Impedance of the WMFR

Two broadband HFSS simulations were implemented in order to determine the complex impedances Zₘₑₛ of two WMFRs under consideration, calculated with no load, i.e. without a container or medium to be investigated in the case of the bench of FIG. 5.

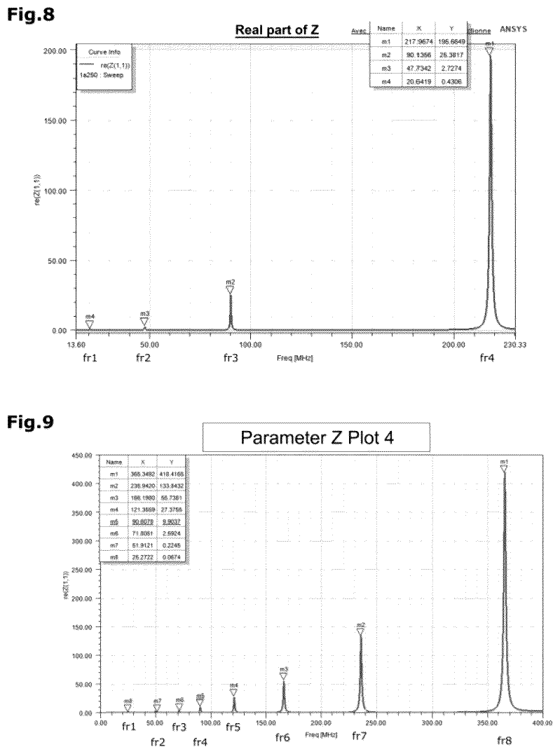

FIG. 8 shows the result for the real part of the measured impedance, according to frequency, for the multifrequency resonator MR1 with four elementary transmission lines (i.e. of WMFR-4 type), of FIG. 1a. The geometric characteristics are those of the four TLRs of batch 1 described above, i.e. M11, M12, M13, M14.

FIG. 9 shows the result for the real part of the measured impedance, according to frequency, for the multifrequency resonator MR2 with eight elementary transmission lines (i.e. of WMFR-8 type), of FIG. 1b. The geometric characteristics are those of the eight TLRs of batch 2 described above, i.e. M21, M22, M23, M24, M25, M26, M27, M28.

It is again seen in these simulations that the resonances of the various TLRs involved are clearly observable with one peak per TLR, and that the association thereof in the form of a WMFR does not jeopardize their individual resonances.

These results show that it is possible to predict the behavior of the multiresonant system formed by the WMFR: the useful information is then extracted from the difference between the impedance with no load and the impedance in the presence of the medium to be investigated.

### Test Bench

FIG. 5 shows a test bench used to implement the two examples MR1 and MR2 of WMFRs with four resonances and eight resonances, illustrated in FIG. 1a and FIG. 1b, respectively, with the same parameters as for the simulations, i.e. a substrate of relative permittivity ∈ᵣ=3.4, a thickness th_{diel}=50 μm, and elementary transmission lines (TLRs) of thickness th_{cond}=35 μm. The inductive loop parameters are the same as above.

The first, WMFR-4, consists of the four MTLRs of batch 1, and the second, WMFR-8, consists of the eight MTLRs of batch 2. The experimental implementation was carried out over a wide frequency band, from a few MHz to 400 MHZ.

Like for the model, the presence of resonance peaks is observed, which can be attributed to each of the TLRs constituting the WMFR. If, moreover, the resonance values observed for the two WMFRs are compared with those provided by the semi-analytical model, SAM (from the matrix of equation (1.3.2.1) which gives equation (1.3.2.3)), and the numerical (HFSS) model, which are presented above, a very good fit between the experimental and modeled values is again observed. Indeed, for WMFR-4, an average deviation of 8% is observed between the data determined by HFSS and those measured, and an average deviation of about 4.5% is observed between the analytical model and the measurement in table 1. For WMFR-8, these average deviations are about 8% and 9%, respectively, in table 2.

FIG. 10 shows, in the form of curves, the comparison of the frequency peaks observed for the various TLRs of each multifrequency resonator MR1 and MR2.

These results show that in practice, it is quite possible to make use of the resonances of the different MTLRs making up the WMFRs. The resonance frequencies observed for the multifrequency resonator are slightly different from the resonance frequencies of its TLRs taken in isolation, due to the couplings that occur between these TLRs.

However, it is quite possible to predict these couplings, via analytical or numerical modeling, and therefore also the resulting resonance frequencies observed for the WMFR.

Furthermore, it will be noted that these couplings do not negatively the resonance properties of the TLRs making up the WMFRs (single-turn in this case): they all have a resonance frequency that can be predetermined, and a quality coefficient close to those measured on the TLRs in isolation. It is therefore entirely conceivable to use the WMFRs as multiple-frequency resonators, in a manner akin to that used for a single TLR for characterizing dielectric media.

For WMFR-4 and WMFR-8, measured with no load, the observed resonance frequencies and characteristics are as follows:

TABLE 3

| | | | | | | | de- | de- |
|---|---|---|---|---|---|---|---|---|
| | | | | | | | via- | via- |
| | | | | | | | tion | tion |
| | Rₑₓₜ | w_{d} | | fₘₑₛ | f_{HFSS} | f_{SAM} | HFSS | SAM |
| Name | [mm] | [mm] | Qₘₑₛ | [MHz] | [MHz] | [MHz] | [%] | [%] |
| M11 | 10.5 | 1 | 270 | 251 | 218 | 256 | 13.2 | 2.0 |
| M12 | 18.75 | 2.5 | 347 | 99.9 | 90.1 | 97.1 | 9.9 | 2.9 |
| M13 | 30 | 5 | 411 | 47.6 | 47.7 | 44.3 | 0.1 | 7.0 |
| M14 | 47.5 | 10 | 565 | 20.9 | 20.6 | 19.7 | 1.5 | 5.9 |

TABLE 4

| | | | | | | | deviation |
|---|---|---|---|---|---|---|---|
| | Rₑₓₜ | w_{d} | | fₘₑₛ | f_{HFSS} | f_{SAM} | HFSS |
| Name | [mm] | [mm] | Qₘₑₛ | [MHz] | [MHz] | [MHz] | [%] |
| M21 | 8 | 1.5 | 355 | 366 | 349 | 3.1 | 1.5 |
| M22 | 11 | 2 | 233 | 236 | 218 | 1.2 | 6.8 |
| M23 | 14.5 | 2.5 | 162 | 167 | 147 | 3.1 | 9.3 |
| M24 | 18.5 | 3 | 115 | 124 | 103 | 7.7 | 10.6 |
| M25 | 23.5 | 4 | 82.2 | 92.2 | 71.5 | 12.2 | 12.9 |
| M26 | 29.5 | 5 | 60.3 | 72.6 | 51.2 | 20.3 | 15.1 |
| M27 | 36.5 | 6 | 43.3 | 50.3 | 38.2 | 16.2 | 11.8 |
| M28 | 44.5 | 7 | 22.3 | 21.6 | 20.8 | 2.8 | 6.7 |

### Method for Characterizing a Dielectric Medium

For the contactless multifrequency characterization of an electrical medium, the implementation consists in arranging the WMFR assembly MR2 and the excitation coil S1 in the vicinity of the dielectric medium 91 to be characterized, and in measuring the impedance Zₘₑₛ seen from the excitation coil using the VNA in a wide frequency band.

FIG. 5 shows the placement of the WMFR in the presence of a homogeneous dielectric medium 91, contained in a container such as a glass dish 81. The excitation coil S1 is installed as a probe. The network analyzer (VNA) S0 is connected to this probe so as to provide it 102 with the excitation current and to measure 104 its impedance.

This characterization of a dielectric medium is possible by virtue of the relationship between the dielectric properties of the medium surrounding the WMFR and the field induced by the WMFR therein. It is established that the complex dielectric properties of organic media are very good indicators of the physical-chemical or physiopathological state thereof, in particular in document [16]. These properties are representative of the way in which the medium dissipates the electromagnetic energy transmitted thereto, through its electrical conductivity σ(in S/m), and the way in which the medium stores the transmitted energy, through its relative dielectric permittivity, ϵr (dimensionless), as described in document [28]. The evaluation of these dielectric properties with frequency, in particular in the radiofrequency range (a few MHz to a few hundred MHz) is particularly relevant when it comes to providing information on the state of organic matter, as described in document [29]. The relationship between the dielectric properties of the medium and the field radiated by the WMFR is expressed through the expression of the impedance Zₘ of the investigated medium, according to document [30]:

$${Z}_{i n} = \int \int {\int }_{medium} {ω}^{2} ( σ + j ω {ε}_{θ} {ε}_{r} ) \frac{{\vec{A}}^{2}}{{\vert I \vert}^{2}} dV \qquad ( 1.3 \text{.5} \text{.1} )$$

where Zₘ corresponds to the impedance of the dielectric medium, σ is the electrical conductivity, ϵᵣ is the relative permittivity, ϵ₀ is the permittivity of free space, Ā is the vector potential associated with the magnetic field generated by the WMFR, I is the current flowing through the WMFR, and w is the angular frequency. Reciprocally, the magnetic field, and therefore the impedance of the WMFR, is modified by the surrounding dielectric medium.

The configuration of FIG. 5 can be represented schematically by the equivalent electrical circuit shown in FIG. 6.

This equivalent diagram groups together the magnetic and electrical interactions taking place between the various elements in association with one another, namely the WMFR MR2 and its various constituent TLRs M21 to M28, the excitation coil S1 connected to the VNA S0, and the dielectric medium 81, itself modeled by a complex impedance Rₘ, Lₘ. As in the case of a TLR interacting with a dielectric medium, the simplified assumption can be made that the excitation coil is not directly coupled to the medium, as described in document [31].

However, each TLR of the WMFR is coupled to the medium through the coupling coefficients kₘⱼ and, as above, to the other TLRs and to the excitation coil. Next, the simplified assumption is also made that the coupling between the various TLRs and the medium can be modeled by a complex impedance Zₘⱼ “induced” by the medium, which is added to the impedance Rⱼ, Lⱼ, Cⱼ in the branch relating to each TLRⱼ, with j∈{1, 2, . . . , N}. In this assumption, the matrix expressing all of the couplings is written as:

$$[ \begin{matrix}{Z}_{S} & j ω {M}_{S 1} & … & j ω {M}_{SN} \\ j ω {M}_{1 S} & {Z}_{1} + {Z}_{{m}_{1}} & … & ⋮ \\ ⋮ & ⋮ & ⋱ & ⋮ \\ j ω {M}_{NS} & j ω {M}_{N 1} & … & {Z}_{N} + {Z}_{{m}_{N}}\end{matrix} ] [ \begin{matrix}{i}_{s} \\ {i}_{1} \\ ⋮ \\ ⋮ \\ {i}_{N}\end{matrix} ] = [ \begin{matrix}{v}_{s} \\ 0 \\ ⋮ \\ ⋮ \\ 0\end{matrix} ] \qquad ( 1.3 \text{.5} \text{.2} )$$

where

$${M}_{ab} = {M}_{ba} and {M}_{ab} = {k}_{ab} \sqrt{{L}_{a} {L}_{b}} for a \neq b and$$

$${Z}_{j} + {Z}_{{m}_{j}} = {R}_{j} + {R}_{{m}_{j}} + j 2 π f ( {L}_{j} + {L}_{{m}_{j}} ) + \frac{1}{j 2 π {fC}_{j}}$$

By inverting this matrix system, for all of the frequencies in the range, it is possible, as above, to extract the impedance Zₘₑₛ. Furthermore, by focusing on a narrow frequency band Bⱼ centered around the resonance frequency of each of the constituent branches of the WMFR, it is easily possible to express the impedance “seen” from the excitation coil, taking into account only the “active” branch in this frequency band; the impedance Zₘₑₛ in the band Bⱼ is then expressed as:

$${Z}_{mes} - {Z}_{S} = \frac{{C}_{j} {k}_{{S}_{j}}^{2} {L}_{S} ( {L}_{j} + {L}_{{m}_{j}} ) {( j 2 π f )}^{3}}{{1 + {C}_{j} ( {R}_{j} + {R}_{{m}_{j}} ) j 2 π f + {C}_{j} ( {L}_{j} + {L}_{{m}_{j}} ) ( j 2 π f ) )}^{2}} \qquad ( 1.3 \text{.5} \text{.3} )$$

In practice, the parameters of the medium Rₘⱼ and Lₘⱼ induced in each branch can be estimated, in each frequency band, using a complex fitting algorithm for fitting between the experimental data for Zₘₑₛ measured in Bⱼ and the model of equation (1.3.5.3) in the same frequency band; the quantities Rⱼ, Lⱼ, and Cⱼ and Zₛ (impedance of the probe alone with no load) having been previously determined. Now, the values of the elements Rₘⱼ and Lₘⱼ are related to the dielectric properties of the medium, i.e. to the electrical conductivity (σ) of the medium on the one hand, and to the dielectric permittivity (ϵᵣ) of the medium on the other hand, at the resonance frequency of the TLR in question, as described in document [12]. By extension, in the case of the WMFR, the extraction of the impedance induced at the N resonance frequencies, using equation (1.3.5.3), makes it possible to deduce therefrom information on the dielectric parameters of the medium, at as many analysis frequencies as there are resonators in the WMFR.

### Characterizing an Aqueous Solution of NaCl

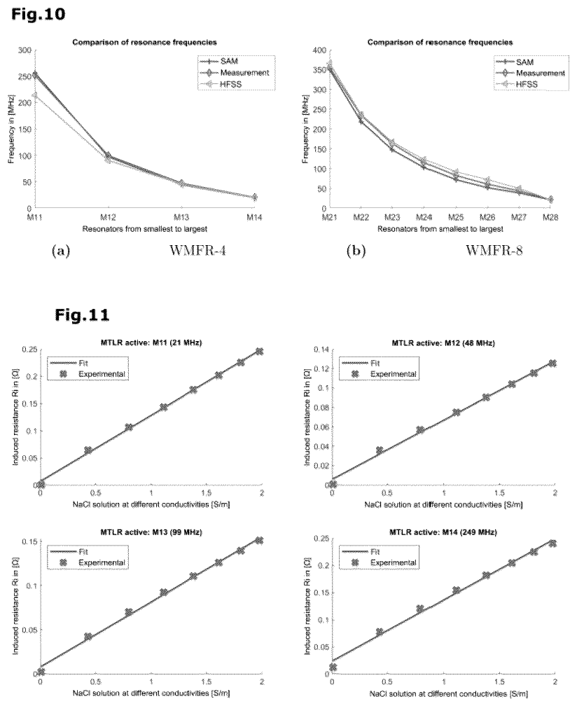

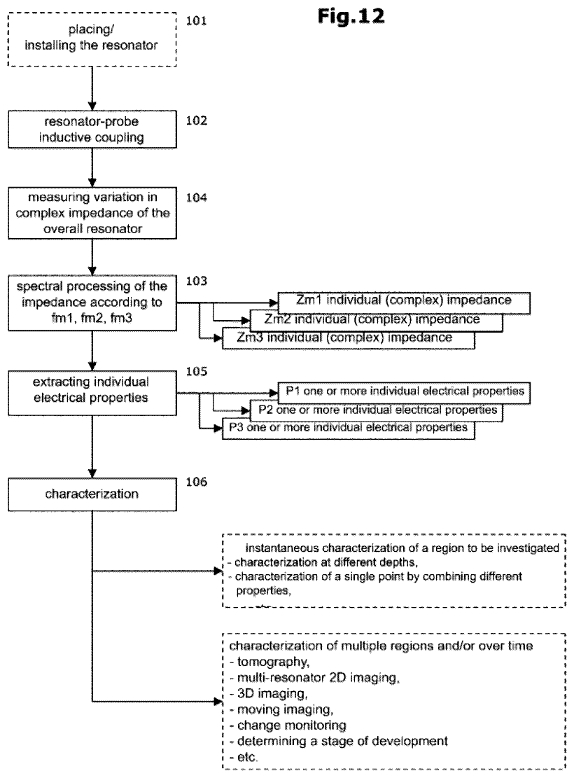

FIG. 11 is an individually shows, for each of the four meshes of the four transmission lines of the four-frequency resonator MR1 of FIG. 1a, the resistance induced according to the conductivity of the medium to be characterized, formed by an aqueous solution of sodium chloride.

The solutions used are conductive saline solutions (deionized water+NaCl) in various proportions, in order to obtain media that have adjustable and tabulated electrical properties (electrical conductivity or dielectric permittivity).

Each of these solutions, with a constant volume of 700 ml, is placed in turn in the glass dish 81. For each solution, multiple measurements of Zₘₑₛ are carried out around each of the resonances of the WMFR.

After implementing the WMFR on all of the calibrated dielectric solutions, the complex impedance induced by the medium (elements Rᵢ et Lᵢ) are estimated around each of the resonance frequencies as explained further in conjunction with equation (1.3.5.3).

For each of the resonance frequencies of the multifrequency resonator, results already observed for just MTLRs are returned: there is, on the one hand, a linearity between the change in the resistance Rᵢ induced in the medium and the electrical conductivity of the medium and, on the other hand, a linearity between the inductance Lᵢ induced in the medium and the dielectric permittivity thereof. These linearities are graphically highlighted in the curves of FIG. 11.

It is thus observed that the WMFR-4 is sensitive, at each resonance, to very small variations in permittivity and conductivity in a homogeneous medium via the extraction of the two parameters of interest, Lᵢ and Rᵢ. Similar results are observed for the WMFR-8.

Thus, a WMFR with N elementary transmission lines forms a device that allows N simultaneous analyses of the medium, enriching the dielectric characterization thereof accordingly.

This invention is therefore particularly advantageous for analyzing organic media, in which it is known that the change with frequency of dielectric parameters of the organic medium is a particularly relevant marker of its physical-chemical state, or physiopathology.

### Studying the Change in a Piece of Meat

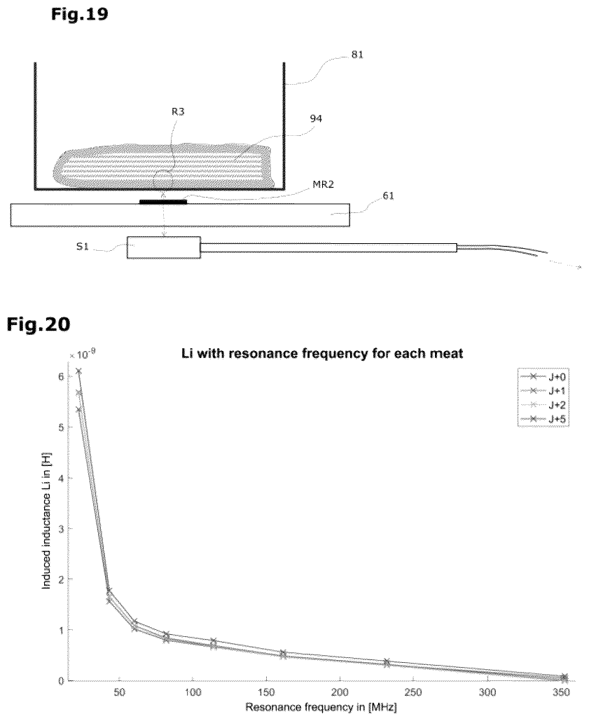

As shown in FIG. 19, such a multifrequency resonator and its individual probe S1 can be used to investigate the electrical properties of a body placed in a container, without contact with or access to the interior of the container, for example as it passes over a resonator MR2 integrated into or attached to a support 61, for example a food product 94 traveling on a conveyor in an agri-food production chain. The ability for contactless investigation at depth thus allows the product 94 to be investigated to a significant depth as illustrated by region R3.

By way of validation, the multifrequency resonator MR2 (WMFR-8) of FIG. 1b was used to study the decomposition of a veal steak (15 cm long, 2 cm thick, 7 cm wide). This steak was placed in the glass dish as shown in FIG. 5, and then Zₘₑₛ was measured at four separate times: day 0, day 1, day 2 and day 5. By the fifth day, the steak was in an advanced state of decomposition with a change in appearance visible to the naked eye: presence of liquid, change in shape and color.

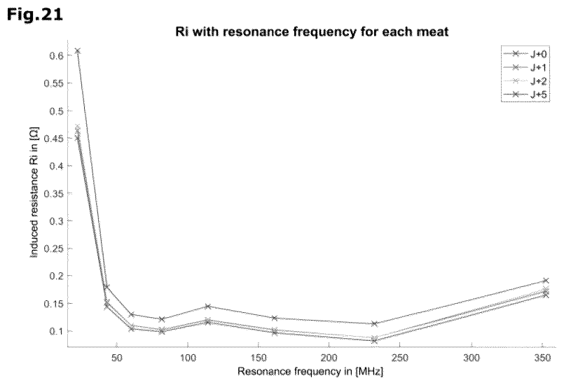

FIG. 20 shows the inductance Lᵢ induced in the steak over time and for the different frequencies, which provide this information for different depths of the region to be investigated R3. FIG. 21 is a similar curve which shows the induced resistance Rᵢ.

By virtue of FIG. 20 and FIG. 21, the advantage of such multifrequency measurement can be seen. Indeed, these two figures make it possible to see the change in the two parameters of interest, Rᵢ et Lᵢ which, as is known, are proportional to σ and ϵᵣ, respectively. It is observed that the dielectric properties of the veal steak vary with the length of decomposition. Indeed, the inductance value Lᵢ induced for each frequency appears to increase with time and with relatively marked differences depending on the frequency. Likewise, for the induced resistance Rᵢ, a very distinct difference is visible between the curve at D+5 and the others, in particular for the lowest frequency 22 MHz and much less for the highest frequency. In addition, from D+1, a variation in Lᵢ and Rᵢ becomes apparent. This becomes more marked with time, and it can be seen that the curve at D+5 is much more distinct from the others. The phenomenon of decomposition can therefore be monitored from the start by the WMFR, and continued to an advanced stage. These results confirm that this WMFR multifrequency sensor is capable of monitoring, in real time, the properties of an organic dielectric medium, non-invasively and contactlessly.

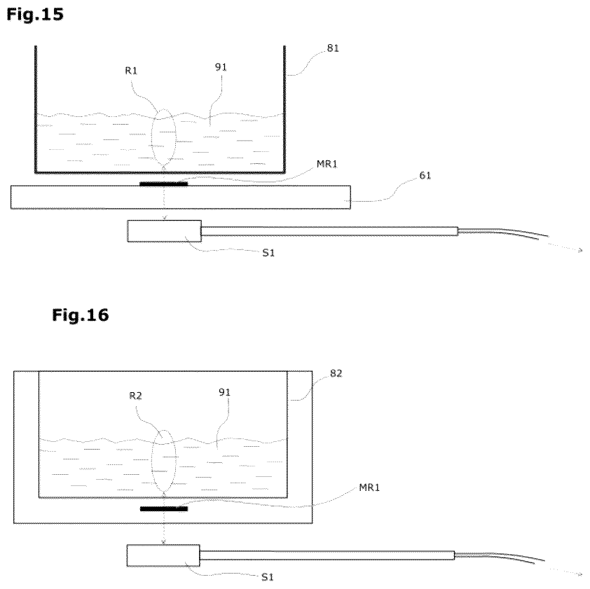

FIG. 12 shows an example of the method for implementing such a multifrequency resonator MR1 for different characterization applications, for example that shown in FIG. 15.

The resonator is placed 101 at the appropriate distance from the region to be investigated, for example removably or implanted within a nearby object. Before or at the time of the measurement, the inductive portion of the probe S1 is arranged at the appropriate distance from the resonator MR1, temporarily or permanently, for example so that it is itself implanted.

The probe S1 is excited by the reader S0 in order to obtain inductive coupling 102 between the probe and the resonator MR1. The reader S0 carries out a measurement 104 of the complex impedance of the assembly formed by the resonator MR1 and the region to be investigated R1.

This complex impedance is processed spectrally to obtain the measured individual impedance Zm1-Zm4 for each of the measurement frequencies fm1-fm4.

For example, by means of the methods described herein in association with those described in the documents listed in the annex or known to a person skilled in the art, the individual electrical properties P1-P4 of the medium in the region to be investigated are extracted 105 from these individual impedances Zm1-Zm4 for the various measured frequencies fm1-fm4.

These individual electrical properties are then used to characterize the medium 91 in the region to be investigated R1, for example based on known data or relationships, or for example by comparing or calibrating with reference data, in particular known data or data recorded for calibration, for example using known statistical methods for processing by neural networks.

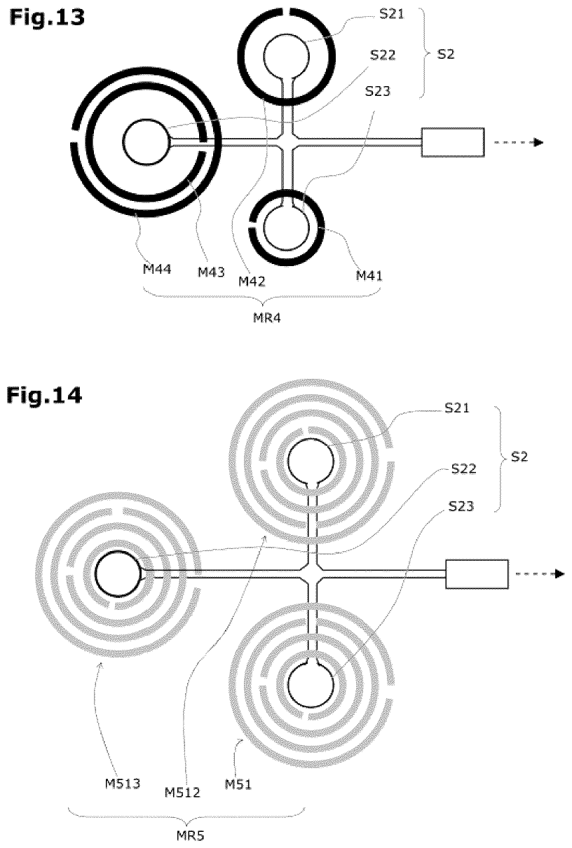

FIG. 13 and FIG. 14 show other exemplary embodiments of the resonator and of the probe with which it is inductively coupled. In these examples, individual transmission lines (TLRs) which are spatially separate and not nested are coupled to the same probe S2.

In this example, the probe S2 comprises a single inductive circuit which is formed of multiple separate portions S21, S22, S23, which could be visually likened to the petals of a daisy.

In FIG. 13, the multifrequency resonator MR4 has two single-turn circular lines M42 and M41 which are separate from one another, and two other single-turn circular lines M 43 and M 44 which are concentric with respect to one another and separate from the other two. These four lines M41-M44, with different individual resonances, are thus spatially divided into three groups, which are typically coplanar, and which can interact with the medium to be investigated in three different subregions. This arrangement makes it possible, for example, to use a large number of different lines without being limited by their size as would be the case if they were all to be arranged concentrically with respect to one another. These different groups are each arranged in the axis of one of the petals of the probe S2, which will then excite them and measure them in a strictly simultaneous manner. For this reason, these four elementary transmission lines together form a single four-frequency resonator MR4, or WMFR-4.

In FIG. 14, the multifrequency resonator MR5 has three groups M511, M512, M513, each with four single-turn circular lines which are different and concentric with respect to one another. These different groups M511, M512, M513 are each arranged in the axis of one of the petals of the probe S2, which will then excite them and measure them in a strictly simultaneous manner. For this reason, these three groups together form a single resonator MR5 with four frequencies, or WMFR-4, if these three groups are identical.

This arrangement then makes it possible to investigate a more extended region with the same frequencies. If the three groups are different from one another, for example in terms of thickness, material or spacing of the tracks, this arrangement also makes it possible to combine a greater number of frequencies; up to twelve frequencies in the present case.

Any type of TLR can be combined in this type of arrangement to produce a single multifrequency resonator, provided that they are coupled to a single same probe and thus that their overall impedance is measured via a single channel.

### Exemplary Implementations

In the example of FIG. 15, the resonator MR1 is used to contactlessly characterize the content 91 of a container 81 that is permeable to the magnetic field, for example a non-magnetic or thin container. In this example, the resonator MR1 is attached to a fixed support 61 over which pass different containers of which the content is to be characterized, for example for quality control or for identification. The resonator makes it possible to contactlessly characterize a region R1 of each of the containers 81 that passes successively thereover.

The example of FIG. 16 shows one implementation in which the resonator MR1 is integrated into the container 82, in this case implanted into its wall. It is thus possible to monitor the region R2 of the content 91, at any time over the course of its change, without needing to open the container 82.

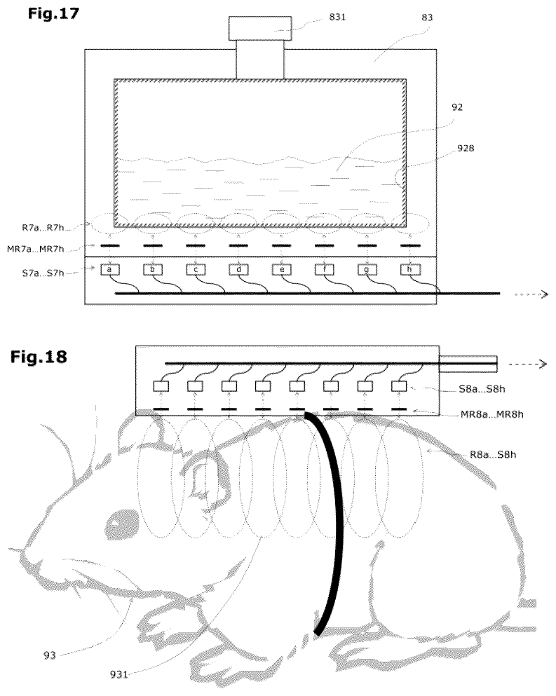

FIG. 17 shows one implementation in which a plurality of multifrequency resonators MR7a to MR7h are implanted in a large number of positions into the wall of a tank, for example a tank 83 for fuel 92, which is closed by a cap 831. These multifrequency resonators are excited by a series of probes S7a to S7h, respectively, which are, for example, integrated into a contiguous plate or even implanted into or attached to the outer wall of the tank. These resonators are sized and positioned so as to characterize the medium present in a series of regions R7a to R7h, for example which encompass the area of interface between the wall of the tank and the fuel. By characterizing these regions of interface in a regular or even permanent manner, it is thus possible to monitor the presence of or change in any bacterial film 928 which develops at any points of corrosion or even promotes it.

FIG. 18 shows one implementation in which a matrix device comprising a plurality of multifrequency resonators MR8a to MR8h, each with its own individual probe S8a to S8h, is placed, in an embedded manner, on a living organism, in this case a mouse 93. Optionally, the probes can also be placed in a separate device, and the resonators can also be implanted directly inside the body of the mouse. Such a system can allow graphical characterization of the change with time of all or part of the body of the mouse to be carried out by virtue of the matrix arrangement of the resonators which each investigate a different region R8a to R8h. It can involve, for example, monitoring and viewing the dispersal throughout the organism of an injected compound, or the change in the state of certain organs over time. Such an embedded configuration allows it to be kept on the mouse throughout the day, and its parameters to be monitored during its waking activities.

### BIBLIOGRAPHY

E. Hammerstad and O. Jensen, “Accurate Models for Microstrip Computer-Aided Design.,” IEEE MTT-S International Microwave Symposium Digest, vol. 1, no. 12, pp. 107-409, 1980, issn: 0149645X. doi: 10.1109/mwsym.1980.1124303.
Y. Luo, X. Wang, and X. Zhou, “Inductance calculations for circular coils with rectangular cross section and parallel axes using inverse mellin transform and generalized hypergeometric functions,” IEEE Transactions on Power Electronics, vol. 32, no. 2, pp. 1367-1374, 2017, issn: 08858993. doi: 10.1109/TPEL.2016.2541180.
B. C. R. Paul, “The concept of “partial” inductance,” pp. 195-245,
J. T. Conway, “Analytical solutions for the self- and mutual inductances of concentric coplanar disk coils,” IEEE Transactions on Magnetics, vol. 49, no. 3, pp. 1135-1142, 2013, issn: 00189464. doi: 10.1109/TMAG.2012.2229287.
G. Masilamany, P. Y. Joubert, S. Serfaty, B. Roucaries, and Y. Le Diraison, “Radiofrequency inductive probe for non-contact dielectric characterizations of organic medium,” Electronics Letters, vol. 50, no. 7, pp. 496-497, 2014, issn: 00135194. doi: 10.1049/el.2014.0558. [Online]. Available: http://digital-library.theiet.org/content/journals/10.1049/el.2014.0558.

1-16. (canceled)

17. A method for characterizing at least one region to be investigated within a medium to be characterized, the method comprising at least the following steps:
contactlessly inductively coupling a probe, simultaneously, to a plurality of transmission lines which are arranged so as to have different resonance frequencies from one another,
said transmission lines together forming a multifrequency resonator, which is located in the vicinity of said investigated region but without requiring contact with said investigated region, the transmission lines of which interact with the region to be investigated: (and typically also with one another)
measuring the variation in impedance of said multifrequency resonator by means of a reader that interacts with said probe;
processing said measurement of variation in impedance, comprising a spectral analysis according to frequency, so as to determine a plurality of individual impedances measured for a plurality of measurement frequencies; and
processing one or more of said individual impedances in order to extract one or more electrical properties of said investigated region.

18. The method as claimed in claim 17, wherein the individual impedances of multiple different resonance frequencies, or the respective electrical properties extracted therefrom, are combined to provide a characterization of the region to be investigated in portions that are located at different distances from the multifrequency resonator.

19. The method as claimed in claim 17, wherein the individual impedances of multiple different resonance frequencies, or the respective electrical properties extracted therefrom, are combined to provide a more accurate characterization of the same portion of the region to be investigated.

20. The method as claimed in claim 17, wherein the method is used to produce a plurality of characterizations at different times, so as to provide monitoring over time of a region to be investigated including at least one material or object undergoing change.

21. The method as claimed in claim 17, wherein the method implements one or more multifrequency resonators which are integrated or implanted into an object or system so as to characterize a region to be investigated,
said region to be investigated including a material belonging to said object, and/or a material in contact with or in the vicinity of said object, and/or an interface between said materials, and
said method being implemented so as to obtain a plurality of time-distributed characterizations for said region to be investigated and thereby provide monitoring over time of a change in the region to be investigated.

22. A system for contactlessly characterizing at least one region referred to as an investigated region within a medium to be characterized, the system comprising:
at least one transmission-line resonator, which is intended to be arranged in the vicinity of said investigated region, but without requiring contact with said investigated region,
a probe arranged so as to:
on the one hand, be coupled via inductive coupling to said resonator by means of an inductive loop circuit, and
on the other hand, to interact with at least one reader;
wherein said resonator comprises a plurality of transmission lines, which are arranged so as to have different resonance frequencies from one another and thereby form a multifrequency resonator, the transmission lines of which interact with the region to be investigated,
and wherein said probe is arranged so as to interact with all of said multifrequency resonator
said reader being arranged so as to interact with said probe in such a way as to implement a method as claimed in claim 17.

23. The system as claimed in claim 22, wherein the multifrequency resonator comprises a plurality of transmission lines which are separate and not connected to one another, and which are each formed by at least one conductive track produced on a two-dimensional dielectric substrate along an almost-closed path.

24. The system as claimed in claim 22, wherein all or some of the transmission lines of the multifrequency resonator each form an almost-closed path with a single turn, typically a single-turn circular split ring.

25. The system as claimed in claim 23, wherein the multifrequency resonator comprises a plurality of conductive tracks of almost-closed shape which are enclosed inside one another.

26. The system as claimed in claim 22, wherein the multifrequency resonator comprises a plurality of transmission lines which are separate and not connected to one another, and which are each formed by a conductive track produced on a two-dimensional insulating substrate, which are in particular paired with one another on both sides of said substrate, and facing and angularly offset, along an “almost-closed” and in particular circular path:
wherein at least two transmission lines of different frequencies are arranged such that they are not nested inside one another, and are referred to as separate;
and wherein the probe comprises an inductive loop circuit, the shape of which is arranged so as to be able to obtain simultaneous inductive coupling to said two separate transmission lines, forming in particular a conductive loop that has single-turn or multiturn projections that are arranged so as to be able to position a projection over each of said separate transmission lines.

27. The system as claimed in claim 22, wherein the multifrequency resonator comprises a plurality of transmission lines which are separate and not connected to one another, and which are formed by conductive tracks each produced along an “almost-closed” path, in a coplanar manner or on the same two-dimensional insulating substrate.

28. The system as claimed in claim 22, wherein the multifrequency resonator comprises a plurality of transmission lines which are separate and not connected to one another, and which are each formed by a conductive track produced on the same two-dimensional insulating substrate, each along a single-turn “almost-closed” path:
and wherein said transmission lines are arranged inside one another, and in particular concentrically with respect to one another.

29. A transmission-line resonator device, of the type capable of communicating via inductive coupling with an inductive loop probe in order to be excited by said probe, so as to interact with said probe,
wherein said device comprises a plurality of transmission lines of almost-closed shape which are enclosed inside one another, in particular concentric circles, and which are arranged so as to form, together, a multifrequency resonator suitable for being implemented within a system as claimed in claim 22.

30. The device as claimed in claim 29, wherein each transmission line forms a single turn.

31. A system comprising an object, said system or object comprising at least one transmission-line resonator, which resonator comprises a plurality of transmission lines which are arranged so as to have different resonance frequencies from one another and thereby form a multifrequency resonator, the transmission lines of which interact with the region to be investigated,
said multifrequency resonator being arranged so as to form an inductive coupling with an inductive loop probe so as to interact with said probe in order to form therewith a system as claimed in claim 22 that is arranged so as to characterize a region to be investigated within a medium to be characterized,
said region to be investigated including a material belonging to said object, and/or a material in contact with or in the vicinity of said object, and/or an interface between said materials.

32. The system as claimed in claim 31, wherein the system comprises at least one probe arranged so as to be able to communicate with the multifrequency resonator via inductive coupling,
and wherein said multifrequency resonator and said probe are arranged so as to form said system to characterize a region to be investigated within a medium to be characterized.
